(19) United States
(12) Patent Application Publication
(10) Pub. No.: US 2014/0330152 A1
(43) Pub. Date: Nov. 6, 2014

(54) PROVIDING EVIDENCE WHETHER AN INTRAVASCULAR CONDUIT IS CORRECTLY POSITIONED

(71) Applicant: Ilan Keidan, Biet Dagan (IL)

(72) Inventor: Ilan Keidan, Biet Dagan (IL)

(21) Appl. No.: 14/006,957

(22) PCT Filed: May 8, 2012
(86) PCT No.: PCT/IB2012/052288

§ 371 Date: Oct. 4, 2013

Related U.S. Application Data
(60) Provisional application No. 61/457,663, filed on May 9, 2011.
(60) Provisional application No. 61/457,766, filed on May 31, 2011.

(51) Int. Cl. A61B 5/06; G01N 33/497; A61B 5/08

(52) U.S. Cl. CPC A61B 5/061; A61B 5/082; G01N 33/497; G01N 2033/4975
USPC 600/532; 436/133

(57) ABSTRACT

Disclosed are methods, devices and compositions useful for providing evidence whether or not an intravascular conduit is correctly positioned in a blood vessel of a living subject, in some embodiments related to administration, through an intravascular conduit, of an indicator precursor such as bicarbonate, and monitoring for the presence of an indicator in the exhaled breath of the subject.

### RELATED APPLICATION

The present application gains priority from U.S. Provisional Patent Applications Nos. 61/457,663 filed 9 May 2011 and 61/457,766 filed 31 May 2011.

### FIELD AND BACKGROUND OF THE INVENTION

The present invention relates to the field of medicine and especially to the field of intravascular administration of compositions to a living subject. Specifically, some embodiments relate to providing evidence whether or not an intravascular conduit is correctly positioned.

It is known to establish an intravascular conduit to allow administration of a composition directly into the blood circulation, typically with the use of a hollow needle, catheter or line. One common type of intravascular administration is intravenous administration.

When the intravascular conduit is in a correct position, an administered composition flows through the conduit directly into the blood circulation, for example, into the lumen of a vein.

Malposition (also called misposition) of an intravascular conduit can occur during the process of establishing the intravascular conduit, especially in emergency medical, geriatric and pediatric (especially neonatal) treatment. Malposition can also occur when an already-positioned intravascular conduit moves from a correct position, for example, when a subject is moved or when a conduit is in place for an extended period of time (hours, days, weeks).

Malposition of an intravascular conduit typically leads to improper administration (incorrect dose, incorrect concentration profile) of the composition as well as extravasation and infiltration. Depending on the nature of the composition, for example when the composition includes vesicant or cytotoxic components, extravasation can potentially cause serious and permanent harm including pain, inflammation and necrosis.

### SUMMARY OF THE INVENTION

Some embodiments of the invention relate to methods, devices and compositions useful in providing evidence whether or not an intravascular conduit is correctly positioned.

In some embodiments, the teachings herein relate to administration, through an intravascular conduit, of an indicator precursor, and monitoring for the presence of an indicator related to the administration in the exhaled breath of the subject. In some embodiments, detection of the indicator related to the administration in the exhaled breath within a time window is accepted as evidence supporting the conclusion that the intravascular conduit is correctly positioned. In some embodiments, failure to detect the indicator related to the administration in the exhaled breath within a time window is accepted as evidence supporting the conclusion that the intravascular conduit is malpositioned.

According to an aspect of some embodiments of the invention, there is provided a method for providing evidence whether or not an intravascular conduit is correctly positioned in the lumen of a blood vessel of a living subject, comprising:

a) through an intravascular conduit in the body of a living subject, administering an indicator composition including an indicator precursor;

b) during a time window, monitoring the exhaled breath of the subject for a presence of an indicator related to the administration of the indicator composition; and

c) accepting as evidence that the intravascular conduit is malpositioned the lack of detection of the presence of the indicator related to the administration of the indicator precursor within the time window.

According to an aspect of some embodiments of the invention, there is also provided a method for providing evidence whether or not an already-positioned intravascular conduit is correctly positioned in the lumen of a blood vessel of a living subject subsequent to administration of an indicator composition including an indicator precursor through the intravascular conduit, comprising:

a) during a time window, monitoring the exhaled breath of the living subject for a presence of an indicator related to the administration of the indicator composition; and

b) accepting as evidence that the intravascular conduit is malpositioned the failure to detect the presence of the indicator related to the administration of the indicator composition within the time window.

According to an aspect of some embodiments of the invention, there is also provided a device useful for providing evidence whether or not an intravascular conduit is correctly positioned in the lumen of a blood vessel of a living subject, comprising:

a digital processor including:
a) an administration signal input for receiving an administration signal that an indicator composition including an indicator precursor is administered to a living subject;
b) an indicator amount input for receiving, from a functionally-associated indicator-determiner, an indicator amount signal indicative of a relative amount of an indicator exhaled by a living subject; and
c) an evidence output;
the processor configured, subsequent to receipt through the administration signal input of an administration signal that an indicator composition including an indicator precursor is administered to a living subject, to accept an indicator amount signal through the indicator amount input over a time window;
and
the processor configured to issue a warning signal through the evidence output if during the time window the indicator amount signal does not indicate exhalation of indicator related to the administration of the indicator precursor, wherein the warning signal indicative that there is evidence that an intravascular conduit is malpositioned.

According to an aspect of some embodiments of the invention, there is also provided an indicator composition useful for providing evidence whether or not an intravascular conduit is correctly positioned in the lumen of a blood vessel of a living subject, comprising:

an indicator precursor; and

a pharmaceutically-acceptable carrier,
the composition configured for intravascular administration; and
the composition configured to lead to the exhalation of a detectable indicator upon intravascular administration to a living subject.

Unless otherwise defined, all technical and scientific terms used herein have the same meaning as commonly understood by one of ordinary skill in the art to which the invention pertains. In case of conflict, the specification, including definitions, take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

Some embodiments of the invention may involve performing or completing selected tasks manually, automatically, or a combination thereof. Some embodiments of the invention are implemented with the use of components that comprise hardware, software, firmware or combinations thereof. In some embodiments, some components are general-purpose components such as general-purpose computers or processors. In some embodiments, some components are dedicated or custom components such as circuits, integrated circuits or software. For example, some embodiments are performed, at least partially, as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network (e.g., Internet, Intranet, wired, wireless) connection. In some embodiments, implementation includes a user interface, generally comprising one or more of input devices (e.g., allowing input of commands and/or parameters) and output devices (e.g., allowing reporting parameters of operation and results).

### BRIEF DESCRIPTION OF THE FIGURES

Some embodiments of the invention are herein described with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a schematic depiction of a first embodiment of a device according to the teachings herein;

FIG. 2 is a schematic depiction of a second embodiment of a device according to the teachings herein;

FIG. 3 is a schematic depiction of a third embodiment of a device according to the teachings herein; and

FIG. 4 is a graph showing the concentration of exhaled CO₂ indicator as a function of the number of exhalations subsequent to administration of an H₂CO₃ composition.

### DESCRIPTION OF SOME EMBODIMENTS OF THE INVENTION

Some embodiments of the invention relate to methods, devices and compositions useful in providing evidence whether or not an intravascular conduit is correctly positioned. In some embodiments, the evidence allows a person to determine whether or not an intravascular conduit is malpositioned.

It will be appreciated that in the priority documents, the term “information” is used as a synonym to the term “evidence” as used herein.

In the context of the teachings herein, correct positioning of an intravascular conduit refers to correct positioning within a blood vessel, such that that fluid flowing through the intravascular conduit enters the lumen of the blood vessel without leaking to surrounding tissue.

In some embodiments, the teachings herein relate to administration, through an intravascular conduit, of an indicator precursor such as bicarbonate, and monitoring for the presence of an indicator (e.g., CO₂) related to the administration of the indicator precursor in the exhaled breath of the subject. In some embodiments, detection of the indicator related to the administration in the exhaled breath within a time window is accepted as evidence supporting the conclusion that the intravascular conduit is correctly positioned. In some embodiments, failure to detect the indicator related to the administration in the exhaled breath within a time window is accepted as evidence supporting the conclusion that the intravascular conduit is malpositioned.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

It is a challenge to ensure that an intravascular conduit is in a correct position in the vasculature of a living subject, to assist in ensuring that it is possible to administer a composition through the conduit, whether during initial establishment of the conduit or subsequently, for example, after the subject has been moved or after an extended period of time subsequent to establishment of the conduit.

In some embodiments the teachings herein provide methods, devices and compositions useful in providing evidence to assist a person, such as medical personnel, in determining whether or not an intravascular conduit is in a correct position in the vasculature of a living subject.

### Methods for Providing Evidence

According to an aspect of some embodiments of the teachings herein there is provided a method for providing evidence whether or not an intravascular conduit is correctly positioned in the lumen of a blood vessel of a living subject, comprising:

a) through an intravascular conduit in the body of a living subject, administering an indicator composition including an indicator precursor;

b) during a time window, monitoring the exhaled breath of the subject for the presence of an indicator related to the administration of the indicator composition; and

c) accepting as evidence that the intravascular conduit is malpositioned upon lack of detection of the presence of the indicator related to the administration of the indicator composition within the time window.

According to an aspect of some embodiments of the teachings herein there is also provided a method for providing evidence whether or not an already-positioned intravascular conduit is correctly positioned in the lumen of a blood vessel of a living subject subsequent to administration of an indicator composition including an indicator precursor through the intravascular conduit, comprising:

a) during a time window, monitoring the exhaled breath of the subject for a presence of an indicator related to the administration of the indicator composition; and

b) accepting as evidence that the intravascular conduit is malpositioned the failure to detect the presence of the indicator related to the administration of the indicator composition within the time window.

In some embodiments, the methods further comprise: d) accepting as evidence that the intravascular conduit is correctly positioned in the lumen of a blood vessel of the subject the detection of the presence of indicator related to the administration of the indicator composition within the time window.

### Subject

The subject is any suitable living subject. In some embodiments, the subject is a human. In some embodiments, the subject is a non-human animal.

In some embodiments, the subject is bedridden. In some embodiments, the subject is unconscious. In some embodiments, the subject is mechanically ventilated. In some embodiments, the subject is under general anesthesia. In some embodiments, the subject is intubated, in some embodiments in an intensive care unit. In some embodiments, the subject is heavily sedated. In some embodiments, the subject is comatose. In some embodiments, the subject is paralyzed. In some embodiments, the subject is undergoing cardiac resuscitation. In some embodiments, the subject is a pre-adult. In some embodiments, the subject is a child. In some embodiments, the subject is an infant (for human subjects, between 28 days and 3 years old). In some embodiments, the subject is a neonate (for human subjects, up to 28 days old). In some embodiments, the subject is a human weighing less than 3500 grams, especially a prematurely-born human.

### Intravascular Conduit

The teachings herein are applicable to any suitable intravascular conduit, including catheters, syringes, needles and lines. That said, in some embodiments, the intravascular conduit is an intravenous conduit and the intravascular administration is intravenous administration.

It has been found that some embodiments, especially when the subject is large (e.g., greater than 40 kg and even greater than 50 kg) are exceptionally effective when the intravascular conduit is an intravenous conduit positioned to be as close as possible to the lungs, e.g., in an arm, in a subclavian vein or in a jugular vein. Although not wishing to be held to any one theory, it is believed that such positioning reduces the effect on post-administration dilution in the body of the subject on the indicator composition, indicator precursor and/or indicator, allowing greater certainty as to the significance of the evidence and/or allowing administration of a lesser amount of indicator composition and/or a lesser amount of indicator precursor.

In this context, in some embodiments the teachings herein are unexpectedly effective when used with a small subject, e.g., less than 40 kg, less than 30 kg, even less than 20 kg and even less than 10 kg. It has been found that, in some embodiments, the time window in which the presence of an indicator related to administration of the indicator composition starts more quickly after administration and is shorter and/or a composition with a lesser concentration of indicator precursor can be used even when the intravascular conduit is positioned remotely from the lungs, for example in a leg of the subject. Although not wishing to be held to any one theory, it is believed that the smaller size of the subject and concomitant smaller vasculature allows the indicator composition, indicator precursor and/or indicator to reach the lungs more quickly and with less absorption in the bodily tissue.

### Indicator Precursor, Indicator and Indicator Composition

As noted above, the teachings herein are based on administration of an indicator precursor to a living subject through an intravascular conduit, which indicator precursor is such that intravascular administration (i.e., through a correctly placed intravascular conduit) leads to exhalation of an indicator related to the administration of the indicator precursor.

By “related to the administration of the indicator precursor” is meant that the identity of detected indicator and/or the timing of detection of the indicator and/or the relative amount of indicator detected can be correlated to the administration of the indicator precursor.

For example, in some embodiments, the indicator is an atypical exhalant, that is to say, is not normally exhaled by a subject, e.g. ¹⁴CO₂, and which detection in exhaled breath is identified as related to the administration of the indicator precursor. In some embodiments the indicator is a typical exhalant, that is to say, is a material that is normally exhaled by a subject, e.g., CO₂, but which detection in exhaled breath in an increased amount during a specified time window is identified as related to the administration of the indicator precursor.

A suitable indicator precursor is an indicator precursor which intravascular administration leads to exhalation of an indicator that is related to the intravascular administration of the indicator precursor, while extravasive administration leads to no substantial exhalation of the indicator or to exhalation of the indicator that is substantially different (e.g., lower intensity, spread out over a different (longer) time period) from exhalation that is related to intravascular administration.

Any suitable indicator precursor may be used in implementing the teachings herein. An indicator precursor is typically a component of a suitable indicator composition.

A suitable indicator composition is a composition that includes the indicator precursor and a suitable pharmaceutically-acceptable carrier, so as to ensure that the indicator precursor is safely administrable through an intravascular conduit. A person having ordinary skill in the art of pharmacology is able to formulate a suitable indicator composition for implementing the teachings herein without undue experimentation.

The Inventor has found that a sufficient amount of bicarbonate (as NaHCO₃) administered intravenously, leads to exhalation of an elevated and easily detectable amount of carbon dioxide subsequent to the administration, for human children typically within 2 to 3 exhalations and for human adults typically within 3 to 5 exhalations. The Inventor has also found that the bicarbonate is quickly cleared from the body of the subject so that the amount of exhaled carbon dioxide returns to normal within the third exhalation after administration is stopped. The Inventor has also found that the amount of exhaled carbon dioxide does not substantially increase when bicarbonate is administered extravascularly.

Suitable indicator composition useful in implementing embodiments of the teachings herein that include bicarbonate are known in the art. For example, it is known to safely administer compositions including sodium bicarbonate and monitor subsequent CO₂ exhalation, such as for the study of metabolism (Elia et al in Proc Nutr Soc 1988, 47, 247-258).

Accordingly, in some embodiments, a suitable indicator precursor comprises bicarbonate (e.g., sodium bicarbonate—NaHCO₃), the exhaled indicator is carbon dioxide and the indicator composition includes the bicarbonate, so the composition including bicarbonate is a CO₂-forming composition. In such embodiments, the relative amount of CO₂ in the exhalation of the subject is monitored subsequent to administration of a sufficient dose of indicator precursor (CO₂-forming precursor) through an intravascular conduit already established in the vasculature of the subject. Observation of an elevated relative amount of CO₂ is evidence indicating that the intravascular conduit is in a correct position in the vasculature of the subject. Failure to observe an elevated relative amount of CO₂ amount is evidence indicating that the intravascular conduit is malpositioned in the vasculature of the subject.

In some embodiments, a suitable indicator precursor comprises isotopically-labelled bicarbonate (e.g., isotopically-labelled NaHCO₃), the exhaled indicator is isotopically-labelled carbon dioxide and the indicator composition includes the isotopically-labelled bicarbonate and therefore is an isotopically-labelled CO₂-forming composition.

In some embodiments, the indicator composition is configured to form ¹²CO₂ as an indicator, e.g., includes NaH¹²CO₃ as an indicator precursor. In such embodiments, monitoring of a relative amount of ¹² CO₂ in the exhalation of the subject is performed using any suitable device. In some such embodiments, monitoring of a relative amount of ¹²CO₂ in the exhalation of the subject is performed using electromagnetic spectroscopy, for example infrared spectroscopy, see for example, Mansfield et al in Phys in Med Bio 1998, 43(5), 1225-1239.

In some embodiments, the indicator composition is configured to form ¹³CO₂ (e.g., includes NaH¹³CO₃ as an indicator precursor) and the indicator monitored is ¹³CO₂, in analogy to the urea breath test known in the art of medical diagnosis. In such embodiments, monitoring of a relative amount of ¹³CO₂ in the exhalation of the subject is performed using any suitable device. In some such embodiments, monitoring of a relative amount of ¹³CO₂ in the exhalation of the subject is performed using electromagnetic spectroscopy, for example infrared spectroscopy. In some such embodiments, monitoring of a relative amount of ¹³CO₂ in the exhalation of the subject is performed using mass spectrometry (e.g., isotope ratio mass spectrometry or mass-correlation spectrometry).

In some embodiments, the indicator composition is configured to form ¹⁴CO₂ (e.g., includes NaH¹⁴CO₃ as an indicator precursor) and the indicator monitored is ¹⁴CO₂, in analogy to the urea breath test known in the art of medical diagnosis. In such embodiments, monitoring of a relative amount of ¹⁴CO₂ in the exhalation of the subject is performed using any suitable device. In some such embodiments, monitoring of a relative amount of ¹⁴CO₂ in the exhalation of the subject is performed using electromagnetic spectroscopy. In some such embodiments, monitoring of a relative amount of ¹⁴CO₂ in the exhalation of the subject is performed using mass spectrometry. In some such embodiments, monitoring of a relative amount of ¹⁴CO₂ in the exhalation of the subject is performed using a radiation, especially beta-radiation, detector, such as a scintillation counter.

### Monitoring of Indicator

An indicator may be monitored in any suitable fashion. Typically, suitable methods of monitoring exhaled indicators include absorbance spectroscopy (especially infrared absorbance spectrometry), emission spectroscopy (especially fluorescence), mass spectrometry and radiation detectors such as scintillation counters.

For example, in embodiments in which carbon dioxide (isotopically-labelled or not) is the indicator, infrared absorbance spectrometry as known in the art of capnography is especially effective in detecting elevated relative amounts of exhaled carbon dioxide following administration of a suitable indicator composition. For example, some embodiments of the teachings herein are implemented using a capnometer, for example a commercially-available capnometer (e.g., from Oridion Capnography Inc., Needham, Mass., USA).

For example, in embodiments in which isotopically-labelled carbon dioxide is the indicator, mass spectrometry using electrical ionization is especially effective in detecting elevated relative amounts of exhaled isotopically-labelled carbon dioxide following administration of a suitable indicator composition.

For example, in embodiments in which radioactive isotopically-labelled carbon dioxide is the indicator, a scintillation counter is especially effective in detecting elevated relative amounts of exhaled radioactive isotopically-labelled carbon dioxide following administration of a suitable indicator composition.

### Clearance Time

It is generally preferred that subsequent to intravascular administration of an indicator composition, the indicator precursor and the indicator clear from the body of the subject as quickly as possible. Such clearance leads to a quickly-identifiable lack of indicator in the exhaled breath upon stopping intravascular administration of the indicator composition (e.g., when an intravascular suddenly becomes malpositioned due to movement of the subject).

Accordingly, in some embodiments, the indicator composition is configured so that indicator related to intravascular administration of the indicator composition is not identified in the exhaled breath of the subject within a clearance time of not more than 40 seconds subsequent to ending intravascular administration of the indicator composition. In some embodiments, the clearance time is not more than 30 seconds, not more than 25 seconds, not more than 20 seconds and even not more than 10 seconds.

In some embodiments, the indicator composition is configured so that indicator related to intravascular administration of the indicator composition is not identified in the exhaled breath of the subject within a clearance time of not more than 8 exhalations subsequent to ending intravascular administration of the indicator composition. In some embodiments, the clearance time is not more than 6 exhalations, not more than 5 exhalations, not more than 4 exhalations and even not more than 3 exhalations.

### Detection Time

In some embodiments it is preferred that the indicator related to intravascular administration of the indicator composition is detectable in the exhaled breath as quickly as possible after intravascular administration of the indicator composition begins.

Accordingly, in some embodiments, the indicator composition is configured so that indicator related to intravascular administration of the indicator composition is identified in the exhaled breath of the subject within not more than 40 seconds from the beginning of intravascular administration of the indicator composition, and in some embodiments, not more than 30 seconds, not more than 20 seconds, not more than 15 seconds, not more than 10 seconds and even not more than 5 seconds.

In some embodiments, the indicator composition is configured so that indicator related to intravascular administration of the indicator composition is identified in the exhaled breath of the subject within not more than 8 exhalations from the beginning of the intravascular administration of the indicator composition, and in some embodiments, not more than 6 exhalations, not more than 5 exhalations, not more than 4 exhalations and even not more than 3 exhalations.

### Administration

In implementing the teachings herein, the indicator composition is administered in any suitable intravascular manner.

### Continuous Administration

In some embodiments, the indicator composition is administered continuously, that is to say, administered through the intravascular conduit continuously throughout a period of time. In such embodiments, the duration of the time window is long (e.g., not less than 60 seconds, but more typically minutes and even hours). In such embodiments, as long as the intravascular conduit is properly positioned, the relative amount of indicator detected in the exhaled breath of the subject is at a substantially constant elevated amount. A drop in the relative amount of detected indicator is evidence indicating the possibility that the intravascular conduit is malpositioned and that the indicator composition is no longer being administered intravascularly.

In some such embodiments, monitoring of the relative amount of indicator in the exhaled breath is continuous. In some such embodiments, monitoring of the relative amount of indicator in the exhaled breath is discontinuous, for example periodically, e.g, at a rate more frequent than once every 60 seconds, once every 30 seconds, or even once every 15 seconds.

### Discontinuous Administration

In some embodiments, the indicator composition is administered through the intravascular conduit discontinuously, that is to say, over a period of time where the teachings herein are implemented on a single subject, the indicator composition is administered during one or more discrete administration events. The duration of a single administration is dependent on factors such as the nature of the indicator, and is readily determined by a person having ordinary skill in the art upon perusing the description herein. For example, in some embodiments when the indicator is an atypical exhalant (e.g., so there is relatively little background interference), the duration of a single administration event is short, in some embodiments not more than 5 seconds, not more than 3 seconds, not more than 2 seconds and even not more than 1 second. For example, in some embodiments when the indicator is a typical exhalant and/or the subject is relatively larger and/or a large amount of indicator composition must be administered, the duration of a single administration event may be longer than 5 seconds. In some embodiments, discontinuous administration is at regular intervals. In some embodiments, discontinuous administration is at irregular intervals.

In some such embodiments, the characteristics of the time window for detection of exhaled indicator (e.g., start time and duration of the time window) are coordinated with the administration of the indicator composition, that is to say, the start time of administration, the administration duration, the clearance time of the indicator and indicator precursor and the detection time are taken into consideration when the characteristics of a time window are determined, to ensure that the expected presence of indicator related to a given administration event falls within the time window.

A person having ordinary skill in the art, upon perusal of the disclosure herein, is able to determine a suitable time window taking the start time of administration, the administration duration, the clearance time and the detection time of a specific indicator composition into consideration.

In such embodiments, detection of indicator related to a single administration event of indicator composition (e.g., in some embodiments a transient increase of a relative amount of indicator and/or in some embodiments a sudden substantial increase of a relative amount of indicator) within the time window associated with the administration event is evidence that the intravascular conduit is properly positioned.

Failure to detect the presence of indicator related to a single administration event of the indicator composition within the time window associated with the administration event is evidence that the intravascular conduit is malpositioned.

In some embodiments, the start of a time window is concurrent with the start of an associated administration event. In some embodiments, the start time of a time window is not more than 5 seconds, not more than 4 seconds, not more than 3 seconds and even not more than 2 seconds from the start of an associated administration event. In some embodiments, the start time of a time window is after a first exhalation subsequent to an associated administration event. In some embodiments, the start time of a time window is immediately after a first exhalation subsequent to an associated administration event. In some embodiments, the start time of a time window is concurrently with a first exhalation subsequent to an associated administration event. That said, in some embodiments the start time of a time window precedes the start of an associated administration event.

In some embodiments, the duration of a time window is not more than 60 seconds, not more than 30 seconds, not more than 20 seconds, not more than 15 seconds and even not more than 10 seconds. In some embodiments, the duration of a time window is measured in units of exhalations, for example, not more than 10 exhalations, not more than 6 exhalations, not more than 5 exhalations, not more than 4 exhalations, not more than 3 exhalations, and even not more than 2 exhalations. In some embodiments, the duration of a time window is not more than 60 seconds, not more than 30 seconds, not more than 20 seconds, not more than 15 seconds and even not more than 10 seconds, from the end of administration of the indicator composition.

For example, for a bicarbonate indicator composition for which intravascular administration over a duration of 3 seconds is expected to lead to exhalation of an elevated relative amount of CO₂ within 12 to 25 seconds (3 to 5 exhalations), a suitable time window starts with the administration and is 35 seconds long.

### Response to Possible Conduit Malposition

An intravascular conduit is used for administration of a composition directly to the vasculature of a subject. If the intravascular conduit is malpositioned, the subject may receive an incorrect dose (e.g., if the intravascular conduit falls from the subjects body, if there is excess resistance to composition flow through the conduit, if effective treatment is contingent on intravascular administration, for example of anesthetic) or the subject may be harmed due to extravasation, especially when the composition includes vesicant or cytotoxic components. Harm can be serious and/or permanent harm including pain, inflammation and necrosis.

Once evidence that the intravascular conduit is malpositioned, in accordance with the teachings herein, is accepted, one or more actions may be taken, in some embodiments, automatically, to assist in potentially reducing at least some of the negative effects of a malpositioned intravascular conduit.

In some embodiments, a person (typically a health-care professional) is provided with the evidence and the person then uses their own judgment in deciding how to react.

In some embodiments the method further comprises, upon accepting evidence that the intravascular conduit is malpositioned, activating an alarm, preferably automatically. An alarm is any suitable alarm, including a visual alarm (e.g., flashing light), an audible alarm (e.g., buzz, beep, siren), and/or a tactile alarm (e.g., vibration). Depending on the embodiment, the alarm is delivered in a location near the subject and/or remote to the subject (e.g., a nurses' station) and/or wirelessly (e.g., beeper, smartphone of a health care professional).

In some embodiments the method further comprises, upon accepting evidence that the intravascular conduit is malpositioned, reducing the rate of administration of a composition (typically a beneficial composition, for example including an active pharmaceutical ingredient) through the intravascular conduit, preferably automatically. In some such embodiments, by reducing the rate is meant that administration of the composition through the intravascular conduit is completely stopped, preferably automatically. Such reduction of rate or stopping of administration potentially prevents harm to the subject.

In some embodiments the method further comprises, upon accepting evidence that the intravascular conduit is malpositioned, cooling at least a portion of the body of the subject, preferably automatically. In some embodiments, the portion of the body of the subject cooled is a portion proximal to where the intravascular conduit enters the body of the subject and/or where the intravascular conduit is deployed in the body of the subject. In some embodiments, such cooling is affected by activation of a cooling element, such as a cooling sleeve. In some embodiments, such cooling is affected by topical application of a cooling composition, such as a cooling spray, cold water, or ice water. In some embodiments, such cooling potentially prevents spreading of a harmful composition (typically a beneficial composition, for example including an active pharmaceutical ingredient), reduces inflammation and/or reduces pain.

Embodiments where such cooling may be exceptionally effective include embodiments where a (beneficial) composition administered through the intravascular conduit includes a chemotherapeutic agent, for example, amsacrine, arsenic trioxide, bleomycine, bortezomib, busulfan, carboplatin, carmustine, cisplatin, cladribine, cytarabine, dacarbazine, dactinomycin, daunorubicin, docetaxel, doxorubicin, epirubicin, fluorouracil, gemcitabine, idarubicin, ifosfamide, irinotecan, mechlorethamine, melphalan, mitomycin, mitoxantrone, paclitaxel, plicamycin, streptozocin, teniposide, thiotepa, topotecan and/or trabectedin.

In some embodiments the method further comprises, upon accepting evidence that the intravascular conduit is malpositioned, heating at least a portion of the body of the subject, preferably automatically. In some embodiments, the portion of the body of the subject heated is a portion proximal to where the intravascular conduit enters the body of the subject and/or where the intravascular conduit is deployed in the body of the subject. In some embodiments, such heating is affected by activation of a heating element, such as a heating sleeve. In some embodiments, such heating is affected by topical application of a heating composition, such as warm water.

Embodiments where such heating may be exceptionally effective include embodiments where a composition administered through the intravascular conduit includes a chemotherapeutic agent, for example, etoposide, oxaliplatin, paclitaxel, vinblastine, vincristine, vindesine and/or vinorelbine.

In some embodiments, upon accepting evidence that the intravascular conduit is malpositioned, a force effective to withdraw (aspirate) fluids from the body of the subject through the intravascular conduit is applied, preferably automatically.

In some embodiments the method further comprises, upon accepting evidence that the intravascular conduit is malpositioned, elevating at least a portion of the body of the subject, preferably automatically. In some embodiments, the portion of the body elevated is an arm or leg where the intravascular conduit enters the body of the subject.

In some embodiments the method further comprises, upon accepting evidence that the intravascular conduit is malpositioned, administering a beneficial composition to the subject. In some embodiments, the beneficial composition is an analgesic. In some embodiments, the beneficial composition is an antidote.

In some embodiments, such administration of a beneficial composition is topical administration. In some embodiments, the portion of the body of the subject to which a beneficial composition is topically administered is a portion proximal to where the intravascular conduit enters the body of the subject and/or where the intravascular conduit is deployed in the body of the subject. Embodiments where such topical administration of a beneficial composition may be exceptionally effective include embodiments where a composition administered through the intravascular conduit includes a chemotherapeutic agent, for example topical administration of dimethylsulfoxide during intravascular administration of carboplatin, cisplatin and/or ifosfamide.

In some embodiments, such administration of a beneficial composition is into the body of the subject. In some embodiments, such administration of a beneficial composition is not-intravascularly, for example is subcutaneous. In some embodiments, such administration of a beneficial composition is intravascularly through the intravascular conduit. In some embodiments, the administration of a beneficial composition is intravascularly through a different intravascular conduit, in some embodiments, an intravascular conduit pre-deployed in the body of the subject. Embodiments where such administration of a beneficial composition may be exceptionally effective include embodiments where a composition administered through the intravascular conduit includes a chemotherapeutic agent, for example: administration of sodium thiosulfate during intravascular administration of carboplatin, cisplatin, dacarbazine, mechlorethamine, mitomycin and/or oxaliplatin; administration of hyaluronidase during intravascular administration of carmustine, docetaxel, etoposide, paclitaxel, teniposide, vinblastine, vincristine, vindesine and/or vinorelbine; and administration of dexrazoxane during intravascular administration of daunorubicin, doxorubicin, epirubicin and/or idarubicin.

### Compositions for Providing Evidence

As noted above, beneficial compositions are typically administered to a subject through an intravascular conduit over a period of time. In some embodiments, the teachings herein are implemented to provide evidence whether or not the intravascular conduit is properly positioned to help ensure that the beneficial composition is properly intravascularly administered. Such implementation is especially important when the beneficial composition is toxic or vesicant and/or where improper position of an intravascular conduit may lead to extravasation.

In some such embodiments, the teachings herein are implemented discontinuously, that is to say, an indicator composition is discontinuously administered through the intravascular conduit while the beneficial composition is continuously administered through the intravascular conduit and the lack of an increase or a transient increase of a relative amount of the corresponding indicator in the exhaled breath in a designated time window is considered to be evidence that the intravascular conduit is malpositioned. In some such embodiments, the teachings herein are implemented continuously, that is to say, an indicator composition is continuously administered together with the beneficial compositions through the intravascular conduit and a drop in the relative amount of a corresponding indicator in the exhaled breath is evidence that the intravascular conduit is malpositioned.

According to an aspect of some embodiments of the teachings herein there is also provided an indicator composition useful for providing evidence whether or not an intravascular conduit is correctly positioned in the lumen of a blood vessel of a living subject, comprising: an indicator precursor; and a pharmaceutically-acceptable carrier, the composition configured for intravascular administration and the composition configured to lead to the exhalation of a detectable indicator upon intravascular administration to a living subject.

In some embodiments, the indicator precursor includes bicarbonate, especially sodium bicarbonate, so that the detectable indicator is CO₂ (a typical exhalant, see above). In some embodiments, the indicator precursor is isotopically-labelled, for example isotopically-labelled bicarbonate, so that the detectable indicator is isotopically-labelled CO₂. In some embodiments, the indicator precursor comprises NaH¹²CO₃ so that the indicator comprises ¹²CO₂. In some embodiments, the indicator precursor comprises NaH¹³CO₃ so that the indicator comprises ¹³CO₂. In some embodiments, the indicator precursor comprises NaH¹⁴CO₃ so that the indicator comprises ¹⁴CO₂.

The concentration of indicator precursor in a composition is easily determined by a person having ordinary skill in the art and is based on factors including the sensitivity of the component used to detect the indicator in the exhaled breath of a subject as well as the characteristics (mass, metabolism) of the subject to which the composition is intended to be administered.

Bicarbonate compositions for intravascular administration, especially bicarbonate compositions including NaHCO₃ (isotopically-labelled or not) are known in the art.

The Inventor has found that extravasated indicator compositions having a concentration of 8.4% (1 meq/mL) NaHCO₃ cause tissue damage. The Inventor has found that extravasated compositions having a concentration of 4.2% (0.5 meq/mL) NaHCO₃ do not cause substantial tissue damage. Accordingly, in some embodiments, an indicator composition comprises NaHCO₃ at a concentration of less than 8.4%, less than 7%, less than 6%, less than 5% and even less than 4.3%.

The Inventor has found that intravascular administration of an indicator composition having a concentration of between 2.1% and 4.2% NaHCO₃ to an adult human allows clear identification of the presence of an indicator (CO₂) related to the administration using a standard operating-theater (infrared) capnometer. Accordingly, in some embodiments an indicator composition according to the teachings herein comprises between 2.1% and 4.2% NaHCO₃, especially when the composition is configured for administration to an adult human.

The Inventor has found that intravascular administration of an indicator composition having a concentration of between 0.5% and 4.2% NaHCO₃ to a human child allows clear identification of the presence of an indicator (CO₂) related to the administration using a standard operating-theater (infrared) capnometer. Accordingly, in some embodiments an indicator composition according to the teachings herein comprises between 0.5% and 4.2% NaHCO₃, preferably between 0.5% and 2.1% NaHCO3,especially when the composition is configured for administration to a human child.

It is important to note, that when isotopically-labelled NaHCO₃ is used as an indicator precursor, far lower concentrations of the isotopically-labelled NaHCO₃ may be used.

In some embodiments, the composition further comprises an active pharmaceutical ingredient in an amount sufficient for providing a beneficial pharmaceutical effect upon intravascular administration to a subject in need thereof. Such embodiments allow a single composition to be administered to serve as a beneficial composition as well as an indicator composition in accordance with the teachings herein. In some such embodiments, the concentration of the active pharmaceutical ingredient as well as other components of the composition (e.g., carrier and adjuvants) are typically substantially the same as prior art compositions including the active pharmaceutical ingredient, but having an indicator precursor added thereto at a sufficient concentration. As administration of such compositions is typically continuous over a relatively long time, in some such embodiments it is preferred that the indicator precursor be an isotopically-labelled indicator precursor as the resulting indicator is more easily detected at low concentrations.

In such embodiments, the composition can include any suitable active pharmaceutical ingredient. That said, in some embodiments, the active pharmaceutical ingredient is selected from the group consisting of vesicant agents and cytotoxic agents.

In some embodiments, the active pharmaceutical ingredient is at least one active pharmaceutical ingredient selected from the group consisting of aminophyllines, chlordiazepoxidem, diazepam, digoxin, nafcillin, nitroglycerine, phenytoin, propylene glycol, sodium thiopental, tetracyclines, total parenteral nutrition, plasma, blood and combinations thereof.

In some embodiments, the active pharmaceutical ingredient is at least one active pharmaceutical ingredient selected from the group of cytotoxic active pharmaceutical ingredients consisting of amsacrine, arsenic trioxide, bleomycine, bortezomib, busulfan, carboplatin, carmustine, cisplatin, cladribine, cytarabine, dacarbazine, dactinomycin, daunorubicin, docetaxel, doxorubicin, epirubicin, etoposide, fluorouracil, gemcitabine, idarubicin, ifosfamide, irinotecan, mechlorethamine, melphalan, mitomycin C, mitoxantrone, oxaliplatin, paclitaxel, plicamycin, streptozocin, teniposide, thiotepa, topotecan, trabectedin, vinblastine, vincristine, vindesine, vinorelbine and combinations thereof

In some embodiments, the indicator composition is configured so that indicator related to intravascular administration of the indicator composition is not identified in the exhaled breath of the subject within a clearance time of not more than 40 seconds subsequent to ending intravascular administration of the indicator composition. In some embodiments, the clearance time is not more than 30 seconds, not more than 25 seconds, not more than 20 seconds and even not more than 10 seconds.

In some embodiments, the indicator composition is configured so that indicator related to intravascular administration of the indicator composition is not identified in the exhaled breath of the subject within a clearance time of not more than 8 exhalations subsequent to ending intravascular administration of the indicator composition. In some embodiments, the clearance time is not more than 6 exhalations, not more than 5 exhalations, not more than 4 exhalations and even not more than 3 exhalations.

In some embodiments, the indicator composition is configured so that indicator related to intravascular administration of the indicator composition is identified in the exhaled breath of the subject within not more than 40 seconds from the beginning of intravascular administration of the indicator composition, and in some embodiments, not more than 30 seconds, not more than 20 seconds, not more than 15 seconds, not more than 10 seconds and even not more than 5 seconds.

In some embodiments, the indicator composition is configured so that indicator related to intravascular administration of the indicator composition is identified in the exhaled breath of the subject within not more than 8 exhalations from the beginning of the intravascular administration of the indicator composition, and in some embodiments, not more than 6 exhalations, not more than 5 exhalations, not more than 4 exhalations and even not more than 3 exhalations.

The term “pharmaceutically acceptable carrier” refers to a carrier or a diluent that does not cause significant irritation to a subject and does not substantially abrogate the activity and properties of the other ingredients. An adjuvant is included under these phrases. The term “excipient” refers to an inert substance added to a composition to further facilitate administration of an active ingredient.

Compositions used in implementing the teachings herein may be formulated using techniques with which one of average skill in the art is familiar in a conventional manner, and generally include combining one or more pharmaceutically-acceptable carriers comprising excipients and adjuvants, which facilitate processing of the active ingredients and indicator precursor into a pharmaceutical composition and/or mixing an amount of the active ingredients and indicator precursor with the other components. Suitable techniques are described in “Remington's Pharmaceutical Sciences,” Mack Publishing Co., Easton, Pa., latest edition, which is incorporated herein by reference. For example, pharmaceutical compositions useful in implementing the teachings herein may be manufactured by one or more processes that are well known in the art, e.g., mixing, blending, homogenizing, dissolving, granulating, emulsifying, encapsulating, entrapping and lyophilizing processes.

Compositions suitable for implementing the teachings herein include compositions comprising active ingredients in an amount effective to achieve an intended purpose (a therapeutically effective amount). Determination of a therapeutically effective amount is well within the capability of those skilled in the art, for example, is initially estimated from animal models such as monkeys or pigs.

### Devices for Providing Evidence

A method according to the teachings herein may be implemented using any suitable device or combination of devices. That said, some embodiments are preferentially implemented using a device according to the teachings herein.

According to an aspect of some embodiments of the teachings herein there is provided a device useful for providing evidence whether or not an intravascular conduit is correctly positioned in the lumen of a blood vessel of a living subject, comprising:

a digital processor including:
a) an administration signal input for receiving an administration signal that an indicator composition including an indicator precursor is administered to a living subject;
b) an indicator amount input for receiving, from a functionally-associated indicator-determiner, an indicator amount signal indicative of a relative amount of an indicator exhaled by a living subject; and
c) an evidence output,
the processor configured, subsequent to receipt through the administration signal input of an administration signal that an indicator composition including an indicator precursor is administered to a living subject, to accept an indicator amount signal through the indicator amount input over a time window;
and
the processor configured to issue a warning signal through the evidence output if during the time window the indicator amount signal does not indicate exhalation of indicator related to the administration of the indicator precursor, wherein the warning signal is indicative that there is evidence that an intravascular conduit is malpositioned.

In some embodiments, the device includes a display functionally associated with the processor, to display an indicator-amount signal to a person. Such a display is typically a visual display (e.g., a display screen known in the art of digital computing). A person, typically a health-care professional, may use the display to monitor the relative amount of indicator exhaled by a subject.

In some embodiments, the processor is further configured to issue a clear signal through the evidence output if during the time window the indicator amount signal indicates exhalation of indicator related to the administration of the indicator precursor, wherein the clear signal is indicative that there is evidence that an intravascular conduit is correctly positioned in the lumen of a blood vessel of a living subject.

### Indicator-Determiner

In some embodiments, the device further comprises an indicator-determiner functionally associated with the processor, configured to monitor a relative amount of an indicator exhaled by a living subject and to provide to the processor an indicator-amount signal indicative of the relative amount. Typically, an indicator-determiner functions continuously or discontinuously (periodically) and provides an indicator-amount signal at a useful rate, typically faster than the rate of exhalation of a subject.

Any suitable indicator-determiner can be used in implementing an embodiment of a device as described herein. In some embodiments, an indicator-determiner comprises a capnometer, for example a modified commercially-available capnometer. In some embodiments, an indicator-determiner is configured to determine the relative amount of indicator using an electromagnetic spectrometer (e.g., absorbance spectrometer such as an infrared spectrometer or emission spectrometer such as a fluorescence meter), a mass spectrometer (e.g., quadrupole or TOF mass spectrometer using electron ionization) or a radiation detector, especially beta-radiation detector such as a scintillation counter.

In some embodiments, the indicator-determiner comprises a capnometer configured to monitor a relative amount of CO₂ as an indicator exhaled by a living subject and to provide to the processor an indicator-amount signal indicative of the relative amount.

In some embodiments, the indicator-determiner is configured to monitor a relative amount of isotopically-labelled CO₂ (e.g., ¹³CO₂ and/or ¹⁴CO₂) as an indicator exhaled by a living subject and to provide to the processor an indicator-amount signal indicative of the relative amount.

### Device Configured for Use During Continuous Administration of Indicator Precursor

As noted above, in some embodiments, the teachings herein include continuous administration of an indicator composition to a subject through an intravascular conduit over a period of time. In such embodiments, the relative amount of indicator in the exhaled breath remains substantially constant throughout the administration if the intravascular conduit is properly positioned and a drop in the relative amount of indicator in the exhaled breath is evidence that the intravascular conduit is malpositioned.

In some embodiments, the administration signal is indicative of a continuous administration of an indicator composition. In some such embodiments, the processor, upon receipt of the administration signal, monitors the indicator amount signals received over time (a pre-programmed time, or until the device is stopped) and issues a warning signal when there is a substantial drop in the relative amount of the indicator in the exhaled breath. In some such embodiments, the time window has a duration of at least 60 seconds after receipt of an administration signal. That said, in some such embodiments, the time window is open-ended, that is starts after receipt of an administration signal and continues until stopped.

In some such embodiments, monitoring of the relative amount of indicator is substantially continuous, e.g., the relative amount of indicator in exhaled breath is determined at least as frequently as every exhalation. In some such embodiments, monitoring of the relative indicator amounts is discontinuous, e.g., the relative amount of indicator in exhaled breath is determined not more frequently than once every 10 seconds.

In some such embodiments, the device further comprises a manually-operable switch functionally associated with the processor, such that operation of the switch generates an administration signal indicative of a continuous administration of an indicator precursor. In such embodiments, a person (e.g., health-care professional) typically activates an administration device for continuous administration of a composition through an intravascular conduit, and then activates the manually-operated switch to generate the administration signal to implement the teachings herein using the device.

In some embodiments, the processor is configured to receive the administration signal from a functionally-associated administration device. In some such embodiments, the processor is configured for functional-association, through the administration signal input, with an administration device configured for continuous administration of a composition through an intravascular conduit. When a functionally-associated administration device is activated to administer a composition, the administration device generates the administration signal to implement the teachings herein using the device.

In some embodiments, the device further comprises an administration component configured for administration of a composition through an intravascular conduit, the device configured to generate an administration signal concurrently with activation of the administration component. In some such embodiments, the device further comprises an administration component configured for continuous administration of a composition through an intravascular conduit, the administration component functionally associated with the processor through the administration signal input. When the device is so activated, the administration component is activated to administer a composition and the administration signal is generated to implement the teachings herein using the device.

### Device Configured for Use During Discontinuous Administration of Indicator Precursor

As noted above, in some embodiments, the teachings herein include discontinuous administration of an indicator composition to a subject through an intravascular conduit over a period of time (typically during continuous administration of a beneficial composition). As long as the intravascular conduit is properly positioned, every administration event of indicator composition leads to an increase (in some embodiments transient increase) of the relative amount of a corresponding indicator in the exhaled breath in the designated time window. Failure to detect such an increase (in some embodiments transient increase) of the relative amount of indicator in the time window is considered to be evidence that the intravascular conduit is malpositioned.

In some embodiments, the administration signal is indicative of an instantaneous administration event of an indicator composition.

In some such embodiments, the processor, upon receipt of the administration signal, monitors the indicator amount signals received during a predesignated time window and issues a warning signal upon failure to detect an an increase (in some embodiments transient increase) of the relative amount of indicator during the time window.

As discussed above, the characteristics of the time window (start time and duration) are dependent on factors such as the nature of the indicator composition, the dose of indicator composition administered, rate of administration as well as subject-dependent factors such as body mass and metabolism. That said, in some typical embodiments, the time window starts within not more than 5 seconds, not more than 4 seconds, not more than 3 seconds and even not more than 2 seconds from receipt of the administration signal, in some embodiments determined with reference to a clock, for example an internal clock of the processor. That said, in some typical embodiments, the time window has a duration of not more than 60 seconds, not more than 30 seconds, not more than 20 seconds, not more than 15 seconds and even not more than 10 seconds, in some embodiments determined with reference to a clock, for example an internal clock of the processor.

In some embodiments, the processor further comprises an exhalation input for receiving an exhalation signal indicating that a subject is exhaling from a functionally-associated exhalation detector. In some embodiments, the device further comprises an exhalation detector functionally-associated with the processor through the exhalation input. In some such embodiments, the device is configured to set the start time and/or duration of a time window with reference to the exhalations of a subject, as described above.

In some such embodiments, the device further comprises a manually-operable switch functionally associated with the processor, such that operation of the switch generates an administration signal indicative of an instantaneous administration event of an indicator precursor. In such embodiments, a person (e.g., health-care professional) typically administers an indicator composition and concurrently (that is to say, more or less at the same time) activates the manually-operated switch to generate the administration signal to implement the teachings herein using the device. Such embodiments are exceptionally useful when a health-care professional wants to ensure that an intravascular conduit is properly positioned prior to administration of a beneficial composition, for example, in the field of anesthesiology.

In some embodiments, the processor is configured to receive the administration signal from a functionally-associated administration device. In some such embodiments, the processor is configured for functional-association, through the administration signal input, with an administration device configured for discontinuous administration of a composition through an intravascular conduit, which, in some embodiments, is repeated discontinuous administration. When a functionally-associated administration device is activated to administer a composition (e.g., manually by a health-care professional, or automatically according to a schedule, e.g., at irregular interval or periodically at regular intervals), the administration device generates the administration signal to implement the teachings herein using the device.

In some embodiments, the device further comprises an administration component configured for administration of a composition through an intravascular conduit, the device configured to generate an administration signal concurrently with activation of the administration component. In some such embodiments, the device further comprises an administration component configured for discontinuous administration of a composition through an intravascular conduit, which, in some embodiments, is repeated discontinuous administration, the administration component functionally associated with the processor through the administration signal input. When the device is so activated, every time the administration component is activated to administer a composition, an administration signal is generated to implement the teachings herein using the device.

### Additional Optional Components of the Device

In some embodiments, the device further comprises an alarm component functionally associated with the processor through the evidence output, configured to automatically issue an alarm upon receipt of a warning signal. An alarm component is any suitable alarm component, including a visual alarm component (e.g., a light configured for flashing), an audible alarm component (e.g., a component configured for sounding a buzz, beep, siren), and/or a tactile alarm component (e.g., a component configured for vibration). In some embodiments, the alarm component is physically located in proximity to the subject. In some embodiments, the alarm component is physically located remote to the subject (e.g., a nurses' station). In some embodiments, the alarm component is in wireless communication with other components of the device, (e.g., beeper, smartphone of a health care professional).

In some embodiments, the device further comprises an administration-slowing component functionally associated with the processor through the evidence output, configured to automatically reduce the rate of (and in some embodiments, to completely stop) administration of a composition through the intravascular conduit, upon receipt of a warning signal. In some embodiments, an administration-slowing component is an electromechanical valve or clamp functionally-associatable with a conduit supplying a composition to an intravascular conduit. In some embodiments, an administration-slowing component is an administration device (e.g., a pump) in which rate of administration can be reduced or stopped upon receipt of a warning signal.

In some embodiments, the device further comprises a cooling component functionally associated with the processor through the evidence output, the cooling component configured to automatically cool at least a portion of the body of a subject upon receipt of a warning signal. Typically such a cooling component is configured for topically cooling a portion of a body of a subject that is proximal to where the intravascular conduit enters the body of the subject and/or where the intravascular conduit is deployed in the body of the subject. In some embodiments, the cooling component comprises a device to apply a topical cooling composition such as a cooling spray, cold water or ice water. In some embodiments, the cooling component comprises a cooling sleeve.

In some embodiments, the device further comprises a heating component functionally associated with the processor through the evidence output, configured to automatically heat at least a portion of the body of a subject upon receipt of a warning signal. Typically such a heating component is configured for topically heating a portion of a body of a subject that is proximal to where the intravascular conduit enters the body of the subject and/or where the intravascular conduit is deployed in the body of the subject. In some embodiments, the heating component comprises a heating element such as a heating sleeve.

In some embodiments, the device further comprises a composition-withdrawing component functionally associated with the processor through the evidence output, configured to automatically withdraw fluids (aspirate fluids) through an intravascular conduit upon receipt of a warning signal. In some such embodiments, the composition-withdrawing component is configured to withdraw fluids for a predetermined time and/or of a predetermined amount of fluid. In some embodiments, a composition-withdrawing component is an administration device (e.g., a pump) which can be reversed to withdraw fluids. In some embodiments, a composition-withdrawing component is a component separate from an administration device.

In some embodiments, the device further comprises an elevating component functionally associated with the processor through the evidence output, configured to automatically elevate at least a portion of the body of the subject upon receipt of a warning signal. In some embodiments, the elevating component is configured so that the portion of the body elevated is an arm or leg where the intravascular conduit enters the body of the subject. In some such embodiments, an elevating component is, for example, a motorized sling or a motorized lifting platform.

In some embodiments, the device further comprises an administration component functionally associated with the processor through the evidence output, configured to automatically administer a composition upon receipt of a warning signal. In some embodiments, during use of the device, such an administration component is charged with a beneficial composition (e.g., an analgesic, an antidote) for administration of the beneficial composition upon receipt of a warning signal so as to at least partially ameliorate the potential negative effects that occur if an intravascular conduit becomes malpositioned.

In some such embodiments, the administration component is configured for topical application of a composition, typically to a portion of a body of a subject that is proximal to where the intravascular conduit enters the body of the subject and/or where the intravascular conduit is deployed in the body of the subject. In some embodiments, the administration component includes a sprayer or dropper.

In some such embodiments, the administration component is configured for application of a composition into the body of a subject, typically to a portion of a body of a subject that is proximal to where the intravascular conduit enters the body of the subject and/or where the intravascular conduit is deployed in the body of the subject. In some embodiments, the administration component is configured for intravascular administration of a composition. In some embodiments, the administration component is configured for subcutaneous administration of a composition.

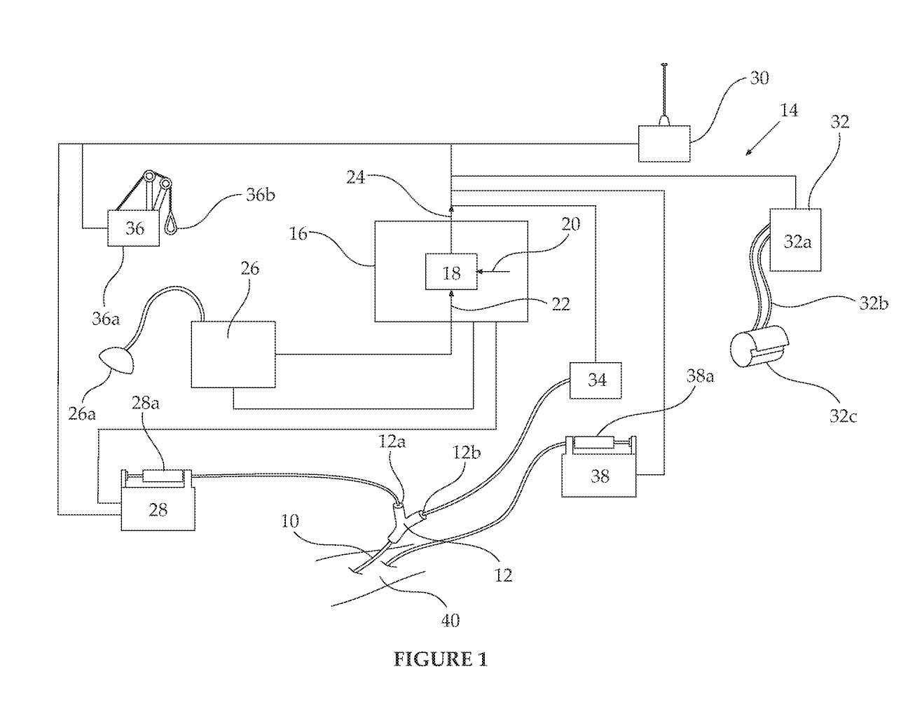

An embodiment of a method according to teachings herein implemented to provide evidence whether or not the intravascular conduit is correctly positioned in the lumen of a blood vessel during the intravascular administration of a chemotherapeutic agent to a subject is described as implemented using an embodiment of a device of the teachings herein with reference to FIG. 1. As is understood by a person having ordinary skill in the art, implementation of the teachings helps reduce the incidence of harm to the subject due to extravasation of a cytotoxic chemotherapeutic agent if the intravascular conduit becomes malpositioned during administration.

In FIG. 1, an intravascular conduit (an intravenous line) 10 is depicted properly positioned in a vein in the arm of a human subject. A T-connector 12 at the proximal end of conduit 10 defines fluid communication between intravascular conduit 10 and inlets 12a and 12b.

Device 14 comprises an adapted general-purpose tablet computer 16 including a digital processor 18 having an administration signal input 20, an indicator amount input 22, and an evidence output 24.

Functionally-associated with processor 18 through indicator amount input 22 is indicator-determiner 26, such as a mass spectrometer, such as an isotope ratio mass spectrometer, for example a MAT 253 isotope mass spectrometer from ThermoFisher Scientific, Waltham, Mass., USA. Indicator-determiner 26 is configured, when activated, to continuously monitor the relative amount of ¹³CO₂ indicator to naturally-occurring ¹²CO₂ entering mask 26a (e.g., by exhalation by a subject wearing mask 26a), to generate an indicator amount signal indicative of the relative amount of ¹³CO₂ indicator, and to provide the indicator amount signal to processor 18 through indicator amount input 22.

A composition administration component 28, e.g., a syringe pump such as a Medfusion® syringe pump available from Smiths Medical, St Paul Minn., USA, is configured to receive commands from computer 16 to administer a composition held in a composition reservoir 28a. Composition administration component 28 is also configured as an administration-slowing component. Specifically, composition administration component 28 is functionally-associated with processor 18 through evidence output 24, and is configured to automatically stop administrating a composition upon receipt of a warning signal from processor 18 through evidence output 24.

Functionally-associated with processor 18 through evidence output 24 is an alarm component 30, which is a communication device configured to automatically wirelessly send an alarm through a Wi-Fi network to a nurses' station (not depicted), upon receipt of a warning signal from processor 18 through evidence output 24.

Functionally-associated with processor 18 through evidence output 24 is a cooling component 32 (e.g., such as described in U.S. Pat. No. 6,402,775) configured to automatically drive cooled fluid from a refrigeration unit 32a, through cooling conduits 32b and into a cooling sleeve 32c, upon receipt of a warning signal from processor 18 through evidence output 24.

Functionally-associated with processor 18 through evidence output 24 and with inlet 12b of T-connecter 12 is a composition-withdrawing component 34 such as an aspirator, for example, a Tao Aspirator™ by Tao & Tao Technology Inc., Camano Island, Wash., USA, configured to automatically withdraw fluid from the body of the subject through intravascular conduit 10 upon receipt of a warning signal from processor 18 through evidence output 24.

Functionally-associated with processor 18 through evidence output 24 is elevating component 36 configured to automatically activate a motor 36a to raise a sling 36b upon receipt of a warning signal from processor 18 through evidence output 24.

Functionally-associated with processor 18 through evidence output 24 is an antidote administration component 38, for example a syringe pump such as a Medfusion® syringe pump configured to automatically begin administration of an antidote composition held in antidote reservoir 38a upon receipt of a warning signal from processor 18 through evidence output 24.

For use, a pharmacist provides an indicator composition according to the teachings herein by adding, to a commercial intravenous cisplatin composition, NaH¹³CO₃ as an indicator precursor in an amount to produce a detectable relative amount of ¹³CO₂ as an indicator, e.g., 0.1% by weight. The pharmacist also provides a 1/6 M sodium thiosulfate solution as an antidote composition

An operator charges composition reservoir 28a of composition administration component 28 with the indicator composition and charges antidote reservoir 38a of antidote administration component 38 with the antidote composition.

The operator establishes fluid communication between the composition outlet of composition administration component 28 with inlet 12a and the aspiration inlet of composition-withdrawing component 34 with inlet 12b, inserts a subcutaneous needle 40 in fluid communication with the antidote outlet of antidote administration component 38 in proximity of where intravascular conduit 10 enters the arm of the subject, places cooling sleeve 32c around the arm above the location where intravascular conduit 10 enters the arm, and then places sling 36b around the arm and cooling sleeve 32c.

The operator places mask 26a of indicator-determiner 26 over the nose and mouth of the subject to capture the exhalations of the subject for analysis by indicator-determiner 26.

The operator initiates continuous administration of the indicator/active pharmaceutical ingredient composition from reservoir 28a using a graphical user interface (GUI) on the touch screen of computer 16 to activate composition administration component 28, initiating continuous cisplatin and indicator precursor administration through intravascular conduit 10. A standard cisplatin administration protocol is followed (e.g., 2 hour or 7 hour duration continuous infusion).

The initiation of administration of the composition activates indicator-determiner 26 to monitor the relative amount of ¹³CO₂ indicator to naturally-occurring ¹²CO₂ exhaled by the subject.

The initiation of administration also generates an administration signal received by processor 18 through administration signal input 20, the administration signal indicative of a continuous administration of an indicator composition to the subject through intravascular conduit 10. Processor 18 defines an open-ended time window during which an indicator amount signal is continuously received from indicator-determiner 26 through indicator amount input 22.

During the administration of the indicator composition, indicator-determiner 26 continuously determines the amount of ¹³CO₂ indicator relative to naturally-occurring ¹²CO₂ in the subject's exhaled breath and provides an indicator amount signal indicative of the amount of ¹³CO₂ indicator relative to naturally-occurring ¹²CO₂ exhaled by a subject to processor 18 through indicator amount input 22. Processor 18 continuously accepts the indicator amount signal through indicator amount input 22.

As long as the relative amount of ¹³CO₂ remains substantially constant at a level greater than normal, processor 18 issues a clear signal through evidence output 24. Upon receipt of a clear signal from processor 18 through evidence output 24, alarm component 30 wirelessly sends a clear signal to a nurses' station, visually displayed as a constantly-lit green light.

At the end of the administration, the subject is disconnected from the various components of device 10.

If during the administration, processor 18 identifies a substantial drop in the relative amount of the ¹³CO₂ indicator to naturally-occurring ¹²CO₂, processor 18 issues a warning signal through evidence output 24.

Upon receipt of a warning signal from processor 18 through evidence output 24, alarm component 30 wirelessly sends an alarm to a nurses' station, visually displayed as a flashing red light, so that health-care professionals at the nurses' station can act in response to the possibility that intravascular conduit 10 is malpositioned.

Upon receipt of a warning signal from processor 18 through evidence output 24, composition administration component 28 stops administration of the indicator composition through intravascular conduit 10.

Upon receipt of a warning signal from processor 18 through evidence output 24, cooling component 32 is activated, driving cooled fluid through cooling sleeve 32c, thereby cooling a portion of the body of the subject in the vicinity of where conduit 10 enters the arm of the subject.

Upon receipt of a warning signal from processor 18 through evidence output 24, composition-withdrawing component 34 is activated, automatically withdrawing fluid from the body of the subject through intravascular conduit 10.

Upon receipt of a warning signal from processor 18 through evidence output 24, elevating component 36 is activated, automatically raising sling 36b and thereby elevating the arm in which intravascular conduit 10 is found.

Upon receipt of a warning signal from processor 18 through evidence output 24, antidote administration component 38 is activated, automatically beginning subcutaneous administration of the antidote solution held in antidote reservoir 38a.

In some related embodiments, cooling component 32 can be used as a heating component to heat a portion of a body of a subject, in a fashion analogous to the cooling described above.

In a related embodiment, a pharmacist provides an indicator composition according to the teachings herein by adding an amount of NaH¹⁴CO₃ and indicator-determiner 26 is replaced with an indicator-determiner comprising a scintillation detector.

Intravenous therapy holds a critical place in modern neonatal care. Intravenous access is often needed immediately after delivery and thereafter for resuscitation, administration of fluids, medications, blood products, and nutrients (e,g., total parenteral nutrition). The lives of prematurely-born, weak or sick neonates are often saved. However, such treatment requires multiple and prolonged intravenous access, meaning that that these most vulnerable of subjects are susceptible to malpositioning of intravascular conduits and concomitant dangers.

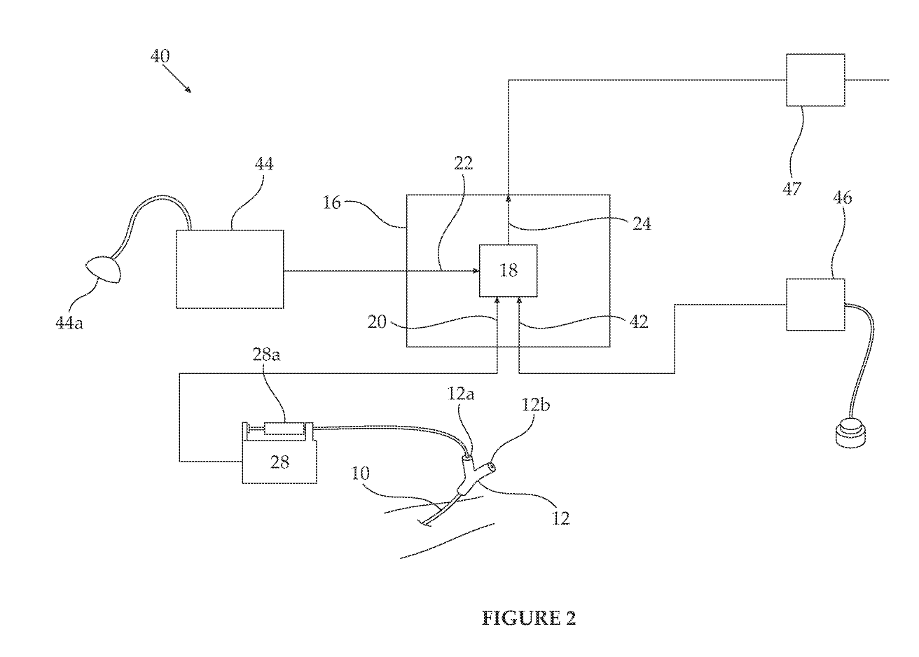

An embodiment of a method according to teachings herein implemented to provide evidence whether or not the intravascular conduit is correctly positioned in the lumen of a blood vessel of a neonate human subject is described as implemented using an embodiment of a device of the teachings herein with reference to FIG. 2. As is understood by a person having ordinary skill in the art, implementation of the teachings helps reduce the incidence of harm to the subject due to extravasation of composition if the intravascular conduit becomes malpositioned.

In FIG. 2, an intravascular conduit (an intravenous line) 10 is depicted properly positioned in a vein of a neonate. A T-connector 12 at the proximal end of conduit 10 defines fluid communication between intravascular conduit 10 and inlets 12a and 12b.

Device 40 comprises an adapted general-purpose tablet computer 16 including a digital processor 18 having an administration signal input 20, an indicator amount input 22, an evidence output 24 and an exhalation input 42.

Functionally-associated with processor 18 through indicator amount input 22 is indicator-determiner 44, such as an infrared capnometer suitable for use with neonates. Indicator-determiner 44 is configured, when activated, to continuously monitor the relative amount of CO₂ indicator entering mask 44a (e.g., by exhalation by a subject wearing mask 44a), to generate an indicator amount signal indicative of the relative amount of CO₂ indicator, and to provide the indicator amount signal to processor 18 through indicator amount input 22.

An exhalation detector 46, such as a pulse oximeter used in accordance with the teachings of Leonard P et al in Emerg Med J 2003, 20, 524-525, is functionally-associated with processor 18 through exhalation input 42. Exhalation detector 46 is configured to detect when a subject exhales, to generate an exhalation signal indicating that the subject is exhaling and to provide processor 18 with the exhalation signal through exhalation input 42.

An indicator composition administration component 28, for example a syringe pump such as a Medfusion® syringe pump available from Smiths Medical, St Paul Minn., USA, is configured to automatically administer a dose (1 ml/kg body weight of the subject) of composition held in a composition reservoir 28a once every five minutes. Composition administration component 28 is also functionally-associated with processor 18 through administration signal input 20 and is configured to send an administration signal to processor 18 through administration signal input 20 concurrently with the start of each administration event.

Functionally-associated with processor 18 through evidence output 24 is an alarm component 47, a component that activates a flashing red light at the bed of the subject and sends an alarm message to a nurses' station (not depicted), upon receipt of a warning signal from processor 18 through evidence output 24.

For use, a pharmacist provides an indicator composition according to the teachings herein, by adding, to a saline composition, NaHCO₃ as an indicator precursor in an amount to produce a detectable relative amount of CO₂ as an indicator, e.g., 1.05% by weight.

An operator charges composition reservoir 28a of composition administration component 28 with the indicator composition.

The operator establishes fluid communication between the composition outlet of composition administration component 28 and intravascular conduit 10 through inlet 12a. Inlet 12b is left free.

The operator places mask 44a of indicator-determiner 44 over the nose and mouth of the subject to capture the exhalations of the subject for analysis by indicator-determiner 44. The operator attaches the sensor of exhalation determiner 46 to the subject.

The operator activates composition administration component 28.

Once every five minutes composition administration component 28 intravascularly administers indicator composition from composition reservoir 28a to the subject through intravascular conduit 10 and sends an administration signal to processor 18 through administration signal input 20 concurrently with the start of each administration event.

The administration signal is received by processor 18 through administration signal input 20, and is indicative of the instantaneous administration event of the indicator composition to the subject through intravascular conduit 10. With reference to the exhalation signals received through exhalation input 42, processor 18 defines a time window starting with the first exhalation following the receipt of the administration signal, and having a duration of three exhalations during which an indicator amount signal related to the administration event is expected.

During the thus-defined time window, processor 18 accepts an indicator amount signal from indicator-determiner 44 through indicator amount input 22.

If, during the time window, an increase in the relative amount of CO₂ is detected in the exhaled breath, nothing is done. At any time, a health-care professional may use inlet 12b for intravascular administration, with increased confidence that conduit 10 is properly positioned.

However, if during the time window no increase in the relative amount of CO₂ is detected, processor 18 issues a warning signal through evidence output 24.

Upon receipt of a warning signal from processor 18 through evidence output 24, alarm component 47 activates the flashing red light and sends an alarm to a nurses' station, so that health-care professionals at the nurses' station can act in response to the possibility that intravascular conduit 10 is malpositioned.

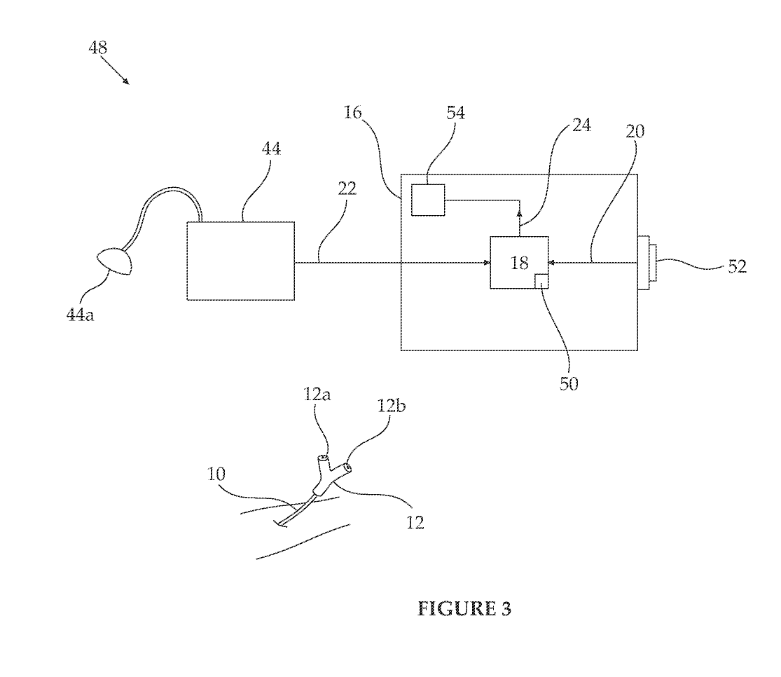

An additional embodiment of a method according to teachings is described as implemented using an embodiment of a device of the teachings herein with reference to FIG. 3, allowing an operator to interrogate whether or not an already-established intravascular conduit is properly positioned.

In FIG. 3, an intravascular conduit (an intravenous line) 10 is depicted properly positioned in a vein of a human subject. A T-connector 12 at the proximal end of conduit 10 defines fluid communication between intravascular conduit 10 and inlets 12a and 12b.

Device 48 comprises an adapted general-purpose tablet computer 16 including a digital processor 18 having an administration signal input 20, an indicator amount input 22, an evidence output 24 and a clock 50.

Device 48 further comprises a manually-operable switch 52 functionally associated with processor 18 through administration signal input 20. Switch 52 is configured to generate an administration signal when manually-operated, and processor 18 is configured to receive the administration signal through administration signal input 20.

Functionally-associated with processor 18 through indicator amount input 22 is indicator-determiner 44, which may be an infrared capnometer. Indicator-determiner 44 is configured, when activated, to continuously monitor the relative amount of CO₂ indicator entering mask 44a, e.g., by exhalation by a subject wearing mask 44a, to generate an indicator amount signal indicative of the relative amount of CO₂ indicator, and to provide the indicator amount signal to processor 18 through indicator amount input 22.

Functionally-associated with processor 18 through evidence output 24 is an alarm component 54, a software component that activates a flashing red light on a portion of the display screen of computer 16 upon receipt of a warning signal from processor 18 through evidence output 24.

For use, a pharmacist provides an indicator composition according to the teachings herein, by adding, to a saline composition, NaHCO₃ as an indicator precursor in an amount to produce a detectable relative amount of CO₂ as an indicator, e.g., 3% by weight.

The operator places mask 44a of indicator-determiner 44 over the nose and mouth of the subject to capture the exhalations of the subject for analysis by indicator-determiner 44.

An operator fills a standard syringe (not depicted) with the indicator composition and establishes fluid communication between the syringe needle and intravascular conduit 10 through inlet 12a. Inlet 12b is left free.

The operator manually operates switch 52, generating an administration signal that is accepted by processor 18 through administration signal input 20, the administration signal indicative of the instantaneous administration event of the indicator composition to the subject through intravascular conduit 10. With reference to clock 50, processor 18 defines a time window starting immediately upon receipt of the administration signal and having a duration of 30 seconds, during which an indicator amount signal related to the administration event is expected.

During the thus-defined time window, processor 18 accepts an indicator amount signal from indicator-determiner 44 through indicator amount input 22.

If during the time window an increase in the relative amount of CO₂ is detected in the exhaled breath of the subject, nothing is done.

However, if during the time window no increase in the relative amount of CO₂ is detected in the exhaled breath, processor 18 issues a warning signal through evidence output 24.

Upon receipt of a warning signal from processor 18 through evidence output 24, alarm component 54 activates the flashing red light.

At any time, a health-care professional may use inlet 12b for intravascular administration.

In some related embodiments, processor 18 is configured to activate a suggestion indicator, e.g., a green light on a portion of the display screen of computer 16, suggesting to a user to implement the teachings herein substantially as described above. In some embodiments, such activation of a suggestion indicator is at regular or irregular intervals. Such embodiments are useful in reminding a health-care professional to implement the teachings herein.

### Experimental

All indicator compositions used were o are prepared by a licensed pharmacist for injection by dissolving NaHCO₃ (as an indicator precursor) in sterile triple-distilled water.

### Safety in Rats

The effect of extravasation of the indicator composition was studied.

Rats were provided and a portion of the skin was shaved of fur.

1.25, 2.5 or 5 ml/kg of 8.4% (1 meq/mL) or 4.2% (0.5 meq/mL) NaHCO3 indicator composition (total 6 animals) or 5 ml/kg of 0.9% saline was administered subcutaneously using a 25 gauge needle under sterile conditions. The rats were observed for one week for signs of local tissue injury resulting from the subcutaneous administration. No substantial injury was observed resulting from the saline injection or from the injection of 4.2% indicator solution. More serious damage (10%) was observed resulting from the injection of 8.4% indicator solution.

### Efficacy in Rat Subjects

1 ml/kg of 4.2% (0.5 meq/mL) NaHCO₃ indicator composition was administered intravenously to 10 rat subjects while the relative amount of CO₂ in the exhaled breath of the subjects was monitored using a capnometer by Oridion Capnography Inc., Needham, Mass., USA. The average baseline relative amount of CO₂ was 33±8 mm Hg which rose to 45±6 mm Hg during intravenous administration.

1 ml/kg of 4.2% (0.5 meq/mL) NaHCO₃ indicator composition was administered subcutaneously to 10 rat subjects while the CO₂ amount of the exhaled breath of the subjects was monitored using the capnometer. The average baseline relative amount of CO₂ was 34±7 mm Hg which remained unchanged at 35±7 mm Hg during subcutaneous administration.

### Efficacy in Human Subjects

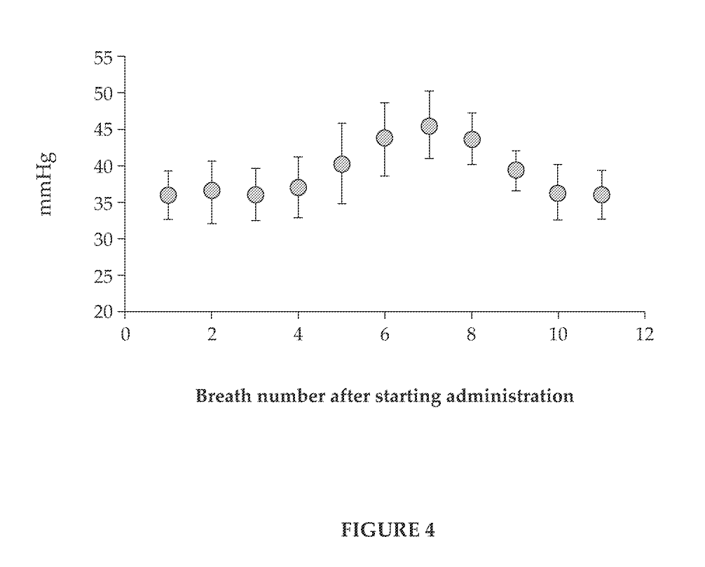

50 ml (−0.75 ml/kg) of 4.2% (0.5 meq/mL) NaHCO₃ indicator composition was administered intravenously through the cephalic vein of the forearm to 20 human subjects over a 3 second period of time while the relative amount of CO₂ in the exhaled breath of the subjects was monitored using a capnometer. The average baseline relative amount CO₂ was 38±5 mm Hg which rose to 45±7 mm Hg during intravenous administration. The relative amount of CO₂ (average of all 20 subjects, mm Hg) in the exhaled breath as a function of time (in units of breaths after administration) is shown in the graph of FIG. 4. From the graph in FIG. 4, it is seen that when using a 4.2% NaHCO₃ indicator composition, a time window of 10 breaths (about 60 seconds) is sufficient to receive evidence whether or not an intravenous conduit is properly positioned in a adult human subject.

50 ml (0.75 ml/kg) of 4.2% (0.5 meq/mL) NaHCO₃ indicator composition was administered subcutaneously to the forearm of 20 human subjects over a 3 second period of time while the relative amount of CO₂ in the exhaled breath of the subjects was monitored using a capnometer. The average baseline relative amount of CO₂ was 36±5 mm Hg which remained unchanged at 35±4 mm Hg during subcutaneous administration.

### Efficacy in Human Children Subjects

The above experiments are repeated for human children aged 3 to 8 years old. It is found that when using a 2.1% or 1.05% NaHCO₃ indicator composition, a time window of 3 breaths (about 10 seconds) is sufficient to receive evidence whether or not an intravenous conduit is properly positioned in a human child subject.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Citation or identification of any reference in this application shall not be construed as an admission that such reference is available as prior art to the invention.

Section headings are used herein to ease understanding of the specification and should not be construed as necessarily limiting.

1. A device to determine whether or not an intravascular conduit is correctly positioned in the lumen of a blood vessel of a living subject, comprising:
a digital processor including:
a) an administration signal input for receiving an administration signal that an indicator composition including an indicator precursor is administered to a living subject;
b) an indicator amount input operably connected to an indicator-determiner configured to generate an indicator amount signal indicative of a relative amount of the indicator composition exhaled by a living subject and to transmit the indicator signal to the digital processor via the indicator amount input; and
c) an evidence output,
said processor configured, subsequent to receipt through said administration signal input of an administration signal that an indicator composition including an indicator precursor is administered to a living subject, to accept an indicator amount signal through said indicator amount input over a time window;
and
said processor configured to (i) determine during said time window from the indicator amount signal whether there is evidence that an intravascular conduit is malpositioned in the body of a living subject and to issue an electronic warning signal through said evidence output in response if during said time window said indicator amount signal does not indicate exhalation of indicator related to said administration of said indicator.

2. The device of claim 1, said processor is further configured to issue a clear signal through said evidence output if during said time window said indicator amount signal indicates exhalation of indicator related to said administration of said indicator precursor, wherein said clear signal is indicative that there is evidence that an intravascular conduit is correctly positioned in the lumen of a blood vessel of a living subject.

3. The device of claim 1, further comprising an indicator-determiner functionally associated with said processor, configured to monitor a relative amount of indicator exhaled by a living subject and to provide said processor with an indicator-amount signal indicative of said relative amount of indicator.

4. The device of claim 3, wherein said indicator-determiner comprises a capnometer configured to monitor a relative amount of CO₂ as said indicator exhaled by a living subject and to provide to said processor a said indicator-amount signal indicative of said relative amount.

5. The device of claim 3, wherein said indicator-determiner is configured to monitor a relative amount of isotopically-labelled CO₂ as said indicator exhaled by a living subject and to provide to said processor a said indicator-amount signal indicative of said relative amount.

6. The device of claim 1, further comprising an alarm component functionally associated with said processor through said evidence output, configured to automatically issue an alarm upon receipt of a said warning signal.

7. The device of claim 1, further comprising an administration slowing component functionally associated with said processor through said evidence output, configured to automatically reduce a rate of administration of a composition through said intravascular conduit upon receipt of a said warning signal.

8. The device of claim 1, further comprising a cooling component functionally associated with said processor through said evidence output, configured to automatically cool at least a portion of the body of a living subject upon receipt of a said warning signal.

9. The device of claim 1, further comprising a heating component functionally associated with said processor through said evidence output, configured to automatically heat at least a portion of the body of a living subject upon receipt of a said warning signal.

10. The device of claim 1, further comprising a composition-withdrawing component functionally associated with said processor through said evidence output, configured to automatically withdraw fluid through said intravascular conduit upon receipt of a said warning signal.

11. The device of claim 1, further comprising an elevating component associated with said processor through said evidence output, configured to automatically elevate at least a portion of the body of a living subject upon receipt of a said warning signal.

12. The device of claim 1, further comprising an administration component associated with said processor through said evidence output, configured to automatically administer a composition upon receipt of a said warning signal.

13. The device of claim 1, wherein said administration signal is indicative of continuous administration of said indicator composition.

14. The device of claim 1, wherein said administration signal is indicative of an instantaneous administration event of said indicator composition.

15. The device of claim 1, further comprising a manually-operable switch, said processor configured to receive said administration signal from said manually-operable switch.

16. The device of claim 1, wherein said processor is configured to receive said administration signal from a functionally-associated administration device.

17. The device of claim 1, further comprising an administration component configured for administration of a composition through an intravascular conduit, the device configured to generate a said administration signal concurrently with activation of said administration component.

18. The device of claim 1, said processor further comprising an exhalation input for receiving an exhalation signal indicating that a living subject is exhaling from a functionally-associated exhalation detector.

19. The device of claim 18, further comprising an exhalation detector functionally-associated with said processor through said exhalation input.

20. An indicator composition useful for providing evidence whether or not an intravascular conduit is correctly positioned in the lumen of a blood vessel of a living subject, comprising:
an indicator precursor; and
a pharmaceutically-acceptable carrier,
the composition configured for intravascular administration; and
the composition configured to lead to the exhalation of a detectable indicator upon intravascular administration to a living subject.

21-28. (canceled)

29. A method for providing evidence whether or not an intravascular conduit is correctly positioned in the lumen of a blood vessel of a living subject, comprising:
a) through an intravascular conduit in the body of a living subject, administering an indicator composition including an indicator precursor;
b) during a time window, monitoring the exhaled breath of the living subject for presence of an indicator related to said administration of said indicator composition; and
c) accepting as evidence that said intravascular conduit is malpositioned the lack of detection of said presence of said indicator related to said administration of said indicator precursor within said time window.

30-51. (canceled)
